(19) United States
(12) Plant Patent Application Publication
(10) Pub. No.: US 2014/0259236 P1
(43) Pub. Date: Sep. 11, 2014

(54) Apple tree named 'REGAL 11-79'

(50) ;

(71) Applicant: Kenneth P. Adams, Ephrata, WA (US)

(72) Inventor: Kenneth P. Adams, Ephrata, WA (US)

(21) Appl. No.: 13/815,571

(22) Filed: Mar. 6, 2013

(51) Int. Cl. A01H 5/00

(52) U.S. Cl. PLT/161

(57) ABSTRACT

A new and distinct variety of apple tree as described and which is characterized as to novelty by producing an attractively colored, small fruit which is mature for harvesting and shipment approximately the last week in August under the ecological conditions prevailing in Central Washington State.

### LATIN NAME

‘Malus domestica Borkh’

### VARIETAL DENOMINATION

‘REGAL 11-79’

### BACKGROUND OF THE NEW VARIETY

The present invention relates to a new and distinct variety of apple tree ‘Malus domestica Borkh’ and which has been denominated varietally, hereinafter, as ‘REGAL 11-79’, and more particularly to an apple tree which bears a small sized, nearly full color fruit which has a melting texture, and a common storage life of nearly three months while remaining crisp, juicy and flavorful, and which further produces leaves which are stipule free, when the tree is grown under the ecological conditions prevailing in Grant County, Washington, in the central region of Washington State.

### ORIGIN

It has long been recognized that an important factor contributing to the success of new varieties of apple trees bearing fruit for the fresh market is its taste, and respective harvesting date in relative comparison to other varieties bearing similar fruit in the same season. Other factors affecting the commercial viability of new strains of apples relates to its ability to remain in cold storage for prolonged periods of time while remaining flavorful and experiencing minimal pressure loss.

The new apple tree ‘Regal 11-79’ is noteworthy in producing fruit, which are ripe for harvesting and shipment, during the last week of August under the ecological conditions prevailing in Grant County, Washington. Further, and in contrast to the apple trees which it is most closely similar to, that being the ‘Honeycrisp’ apple tree, U.S. Plant Pat. No. 7,197, the present tree is distinguished therefrom by being ripe for harvesting and shipment more than two weeks earlier than the ‘Honeycrisp’ apple trees which are grown at the same geographical location. Additionally, and in contrast to the apple tree variety ‘Cripps Pink Cultivar,’ U.S. Plant Pat. No. 7,880, the present variety is ripe for harvesting and shipment approximately six weeks earlier when this patented variety is grown under the ecological conditions prevailing in Central Washington State.

The new variety of apple tree, as described herein, was developed when I made a cross of the ‘Honeycrisp’ apple tree, U.S. Plant Pat. No. 7,197, with the ‘Cripps Pink Cultivar,’ U.S. Plant Pat. No. 7,880, in 2004. The fruit produced from the seedlings of this original cross were first observed during the 2007 growing season. At that moment, the new variety ‘Regal 11-79’ was selected for further observation and cultivation. Thereafter, second generation trees were established by removing bud wood from the promising seedling ‘Regal 11-79’ and budding it into ‘M-26’ root stock [unpatented] and which was then growing in our experimental orchard which is located in Grant County, Washington. After this event, the trees and fruit produced by the second generation trees were first observed in 2009, and were then compared and contrasted against the original seedling found in our experimental orchard.

It has been confirmed through all the aforementioned observations that the earlier asexual reproductions as noted, above, resulted in apple trees which produced fruit, which possessed the same distinct characteristics as the newly discovered tree.

### SUMMARY OF THE NEW VARIETY

The ‘Regal 11-79’ is uniquely distinctive from that of either of its parents, that being, the ‘Honeycrisp’ apple tree (U.S. Plant Pat. No. 7,197), and the ‘Cripps Pink Cultivar’ (U.S. Plant Pat. No. 7,880). The new variety is a spur-type and is very precocious. Further, the new variety is a heavy bearer of fruit that does not exhibit alternative bearing, nor does it exhibit excessive growth. Additionally, the present variety is stipule free. The new variety is an early, mid-season apple, which has dates of harvesting much earlier than either of its parents. The fruit of the new variety is small in size, and exhibits nearly full color as a blush. Additionally, it has been observed that the flesh of the fruit produced by the new variety of apple tree resists browning following exposure of the flesh to the atmosphere, and has a melting texture that is considered to be distinctively snappy, crunchy and juicy, and which further has a noticeable apple-like flavor with an acid level similar to the fruit produced by the ‘Cripps Pink Cultivar’ (U.S. Plant Pat. No. 7,880). Additionally, the fruit produced by the new variety of apple tree has a sugar and flesh texture similar to the fruit produced by the ‘Honeycrisp’ apple tree, (U.S. Plant Pat. No. 7,197). The present variety has a common storage life of nearly three months with minimal pressure loss. The new variety does not exhibit the physiological disorders of Bitter Pit and Lenticel Breakdown that its parent (‘Honeycrisp’, U.S. Plant Pat. No. 7,197) occasionally suffers from.

### BRIEF DESCRIPTION OF THE DRAWINGS

FIG. 1 is a picture of the present variety of apple tree growing under typical conditions, and in its dormant state, in Central Washington State. The tree, as seen in this view, is a second generation, four year old tree.

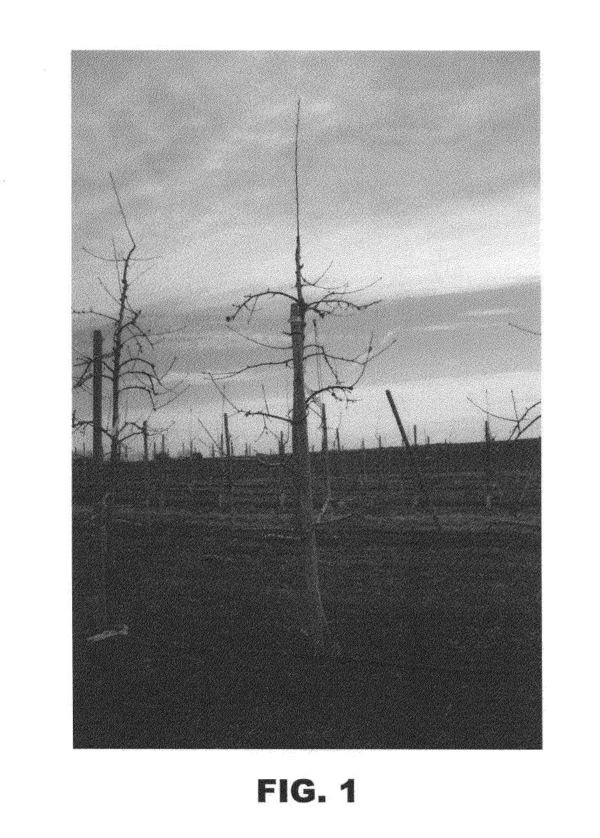

FIG. 2 is a view of a blossom cluster as seen on second generation, four year old trees of the present variety.

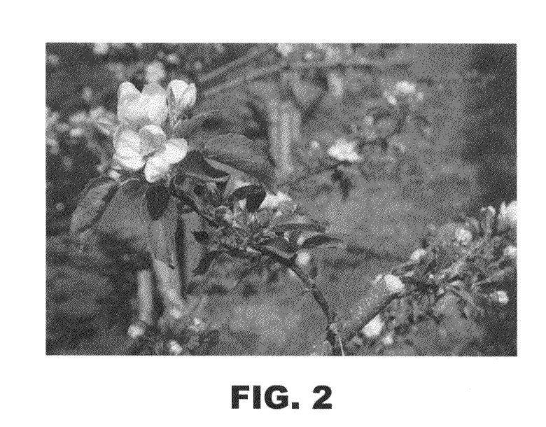

FIG. 3 is a picture of the present variety of tree at full harvest maturity.

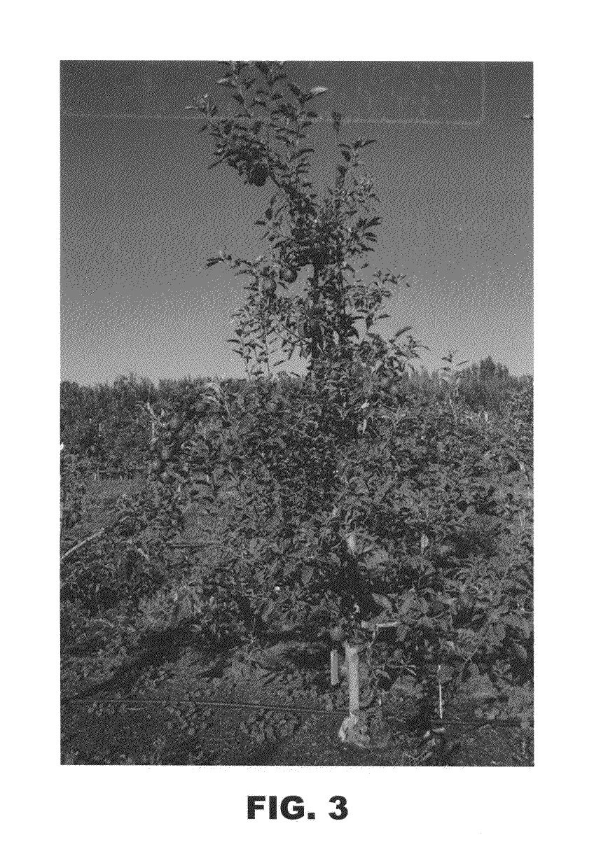

FIG. 4 is a picture of several fruit of the present variety, at full harvest maturity, and in various orientations to illustrate the exterior appearance of the fruit.

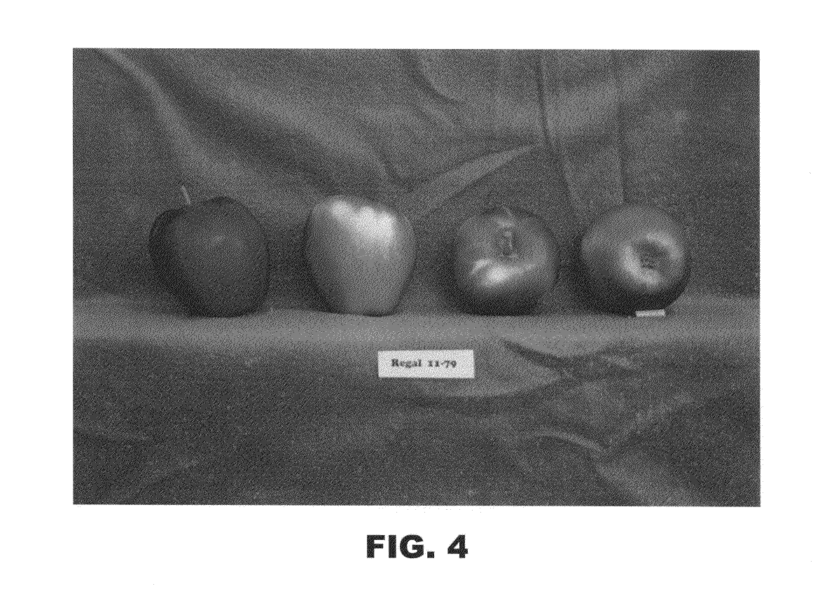

FIG. 5 is a photograph which illustrates the exterior appearance of the fruit produced by the new apple tree in relative comparison to the fruit produced by the parents, that being, the ‘Honeycrisp’ apple tree, (U.S. Plant Pat. No. 7,197), and the ‘Cripps Pink Cultivar’, (U.S. Plant Pat. No. 7,880) at harvest maturity.

The colors in the enclosed photographs are as nearly true as is reasonably possible in color photographs of this type. Due to chemical development, processing, and printing, the various colors depicted in these photographs may, or may not be accurate when compared to the actual botanical specimens. For this reason, future color references should be made to the color plates provided in The Royal Horticultural Society Handbook, and descriptions provided, hereinafter. Occasionally common color names will also be used.

### NOT A COMMERCIAL WARRANTY

The following detailed description has been prepared to solely comply with the provisions of 35 U.S.C. §112, and does not constitute a warranty, (either expressed or implied), that the present variety will, in the future, display the botanical, pomological or other characteristics as set forth, hereinafter. Therefore, this disclosure may not be relied upon to support any future legal claims, including, without limitation, any claims relating to the breach of warranty of merchantability, or fitness for any particular purpose, or non-infringement, which is directed, in whole, or in part, to the present variety.

### DETAILED DESCRIPTION

Tree:
   Size.—The tree, as described hereinafter, is a second generation, asexually reproduced tree, which is four years old. As a general matter, the present tree is average in size, and pruned so as to benefit from a central leader-type cultural arrangement.
   Fruit bearing.—The present variety is considered a consistent bearer with moderately heavy spur development.
   Vigor.—Moderate for the species.
   Overall shape.—Upright, and spreading as trained to a slender spindle.
   Height.—As trained, 13 feet.
   Tree width.—As trained, 7 feet.
   Hardiness.—Considered hardy for USDA Hardiness Zone 6(b).
   Fruit productivity.—Considered moderately heavy for the species.
Trunk:
   Size.—The diameter of the trunk when measured at a height of about 20 cm above the graft union is about 45.6 cm.
   Trunk bark texture.—Smooth.
   Trunk bark color.—Considered gray-orange, [RHS 176B].
   Trunk bark lenticels.—Present, moderate in number, and averaging about 9 lenticels per four square centimeter area.
   Trunk bark lenticels.—Average Length. — About 4.7 mm.
   Trunk bark lenticels.—Average Width — About 0.5 mm.
   Trunk bark lenticels.—Color — White (RHS 155 D).
Branches:
   First year branches.—Diameter — When measured at the midpoint of growth, the first year branches have a diameter of about 5.1 mm.
   First year branches.—Length — About 28.3 cm.
   First year branches.—Color — Gray-purple, (RHS 183 A).
   First year branches.—Lenticels — Numbers — Numerous and averaging about 18 lenticels per running cm of growth.
   First year branches.—Lenticels. — Shape — Round, and about 0.5 to about 1 mm in diameter.
   First year branches.—Lenticels. — Color — White (RHS 155 D).
   First year branches.—Branch Pubescence — Considered moderately dense and covering about 30% to about 50% of the branch surface area.
   First year branch pubescence.—Color — White (RHS N 155 D).
   First year branches.—Internodes — The distance between the internodes ranges from about 26.7 mm to about 33 mm.
Two year old fruiting branches:
   Diameter.—When measured at the midpoint of growth, these branches are about 6.7 mm.
   Two year old branches.—Bark Color — Gray-purple (RHS 187 A).
   Two year old branches.—Spur Development — Considered moderately heavy. The spurs range in length from about 6.4 mm to about 16 inches in length.
   Two year old branches.—Lenticels — The lenticels are moderate in number and average about 8 per square cm of surface area. The lenticels are round, and have a diameter of about 1 to about 1.5 mm.
   Lenticels.—Color — White (RHS 155 D).
   Two year old branches.—Buds — Generally speaking, the buds range in length from about 7.7 mm to about 8.7 mm. The diameter of the buds ranges from about 3.4 mm to about 4.5 mm.
   Bud scale color.—Gray-purple (RHS 187 A).
   Scaffold branches.—Size — These branches range in size from about 21.1 mm to about 22.9 mm, in diameter, when measured at a distance of about 5 cm from the trunk.
   Scaffold branches.—Crotch Angle. — When trained in a central leader arrangement, the crotch angle is about 60° to 90° when measured from the vertical plane.
   Scaffold branches.—Color — Gray-orange (RHS 177 A).
   Scaffold branches.—Lenticels — Generally speaking, they are numerous in number, and average about 6 per square cm of surface area.
   Lenticels.—Shape — Elongated and small. The lenticels range in size from about 1.5 to 2.5 mm in width; and about 0.8 to about 1.2 mm in length.
   Lenticels.—Color — White (RHS 155 D).
Leaves:
   Leaf shape.—Generally speaking the leaves are elliptical.
   Leaf texture.—The dorsal surface is leathery, and the veins appearing on the dorsal surface appear slightly sunken.
   Leaf texture.—Ventral Surface — Glabrous, and the veins appearing on the ventral surface appear to protrude.
   Leaf sheen.—A high surface sheen appears on the dorsal surface.
   Leaf pubescence.—This characteristic is present on the ventral surface only. The pubescence is fine, and light in density, and covers substantially 100% of the ventral surface area.
   Leaf pubescence.—Color — White (RHS 155 C).
   Leaf length.—The leaf blade ranges in length from about 80.2 mm to 100.2 mm.
   Leaf width.—About 41.6 mm to about 56.2 mm.
   Leaf marginal edge.—Crenate.
   Leaf tip.—Shape — Acuminate.
   Leaf base.—Shape — Rounded.
   Leaf stipules.—None appear.
   Leaf color.—The dorsal surface is yellow-green (RHS 147 A). The ventral surface color is yellow-green (RHS 147 B).
   Mid-vein.—Shape — Considered prominent, and having a considerable amount of downiness over the entire surface.
   Mid-vein.—Diameter — When measured at mid-blade it averages about 1.7 mm.
   Mid-vein color.—Ventral Surface — Yellow-green (RHS 150 D).

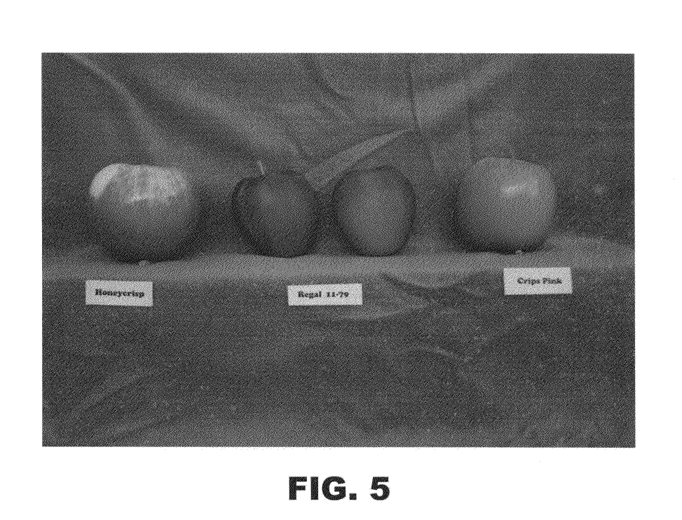

Mid-vein pubescence.—Present, and is white in color (RHS N 155 C).
Leaf petiole.—Length — About 24.7 to 28.7 mm. A shallow groove runs the entire length of the petiole.
Petiole.—Diameter — About 1.6 to about 2.2 mm.
Petiole.—Color — Dorsal Surface — Green (RHS 139 D).
Petiole.—Color — Ventral Surface — Yellow-green (RHS 145 D). The basal end of the petiole may be highlighted with the color red (RHS 46 A).
Petiole pubescence.—General speaking the pubescence is abundant, and has a moderate density over the entire length and circumference thereof. The pubescence color is white (RHS 155 C).
Flowers:
   Date of full bloom.—On average the date of full bloom is sometime between May 1 and May 5 under the ecological conditions occurring in Grant County, Washington State.
   Number of blossoms per bud.—Generally 5 to 6 will be found.
   Flower size.—Generally — Considered medium in size for the species.
   Flower diameter.—When fully expanded the flowers have a diameter of about 45 to about 48.8 mm.
   Flower petal numbers.—5 per blossom.
   Flower petal shape.—Short and elliptical, and having a round base and an obtuse apex.
   Flower petal width.—About 17.1 mm.
   Flower petal length.—About 21.5 mm.
   Flower petal.—Color — When unopened, the petal color is red-purple (RHS 60 B). When opened, the flower petals are white (RHS 155 A). The petals further have red-purple highlights (RHS 60 B).
   Flower stamens.—Numbers — 20.
   Filament.—Length — About 4.1 to about 9.5 mm.
   Filament.—Color — Yellow-green (RHS 150 D).
   Anthers.—Shape — Kidney-like.
   Anthers.—Width — Average width of about 1.5 mm.
   Anthers.—Length — Average length of about 1.8 mm.
   Anthers.—Color — Yellow-green (RHS 150 D).
   Pistil.—Length — About 11.8 mm to about 13.8 mm.
   Styles.—Number — Typically five.
   Styles.—Form — Fused near the base. There is no pubescence present at the union.
   Styles.—Color — Yellow-green (RHS 145 B).
   Stigma.—Shape — Club like.
   Stigma.—Color — Yellow-green (RHS 145 D).
   Pollen production.—A moderate amount of pollen is present.
   Pollen color.—Yellow-green (RHS 145 C).
   Sepals.—Generally — Five sepals appear per blossom, and are curled back towards the peduncle.
   Sepals.—Shape — Considered lanceolate.
   Sepals.—Tip — Generally considered acuminate, with the base being truncate.
   Sepals.—Length — About 10 mm.
   Sepals.—Width — About 3.8 mm.
   Sepals.—Pubescence — The sepal's pubescence is white, and is present on both the upper and lower surfaces.
   Sepals.—Color — Yellow-green (RHS 146 D).
   Peduncle.—Length — About 26 to about 33.2 mm.
   Peduncle.—Diameter — About 1.2 to about 1.4 mm.
   Peduncle.—Color — Yellow-green (RHS 144 A). However, colored highlights appearing on the surfaces exposed to the sun are considered gray-red (RHS 178 A). A considerable amount of white downiness is present over the entire surface. This color is not distinctive of other variety.
Fruit:
   Maturity when described.—The fruit produced by the new, and novel variety of apple tree is described at full commercial maturity. These measurements were taken from second generation trees presently growing in the test orchard which is located in Grant County, Washington.
   Date of harvest.—About Aug. 29, 2012, under the ecological conditions prevailing in Grant County, Washington State. In relative comparison to its two parents, the fruit produced by the ‘Cripps Pink Cultivar’ (U.S. Plant Pat. No. 7,880) matures for harvesting and shipment at the same geographical location on or about Oct. 15, 2012. In further contrast to the other parent, that being the ‘Honeycrisp’ apple tree (U.S. Plant Pat. No. 7,197), this apple tree is mature for harvesting and shipment about September 17^{th} under the ecological conditions prevailing in Grant County, Washington.
   Fruit form.—Generally — Considered uniformly conical. The fruit is generally symmetrical and occasionally lop-sidedness is exhibited. Please see FIG. 5. The horizontal section of the fruit is typically ribbed.
   Fruit size.—Considered small under typical crop loads. On average, the fruit is 6.6 cm in diameter when measured in the equatorial plane, and about 6.4 cm diameter when measured in the axial plane.
   Fruit stem.—Generally — Considered long. The stem length ranges from about 2.5 cm to about 3.2 cm.
   Fruit stem.—Diameter — When measured at mid-length, about 1.8 mm to about 2.7 mm. Occasionally light clubbing is seen.
   Stem color.—Green (RHS 139 C).
   Stem pubescence.—Considered light, and covers about 100% of the surface area.
   Stem pubescence.—Color — White (RHS 155 C).
   Stem cavity.—Generally — About 2.7 cm in width, and having an average depth of about 1.7 cm.
   Stem cavity.—Shape — Considered acute and no russeting is present.
   Stem cavity.—Lipping — None is evident.
   Basin cavity.—Generally — This feature has an average width of about 2.1 cm, and an average depth of about 0.8 cm. The basin cavity is considered more abrupt, than wide, and ribbing is evident. Further light puckering may be observed.
   Eye.—Generally — Reflexed tips are present.
   Eye.—Surface Texture — Moderate downiness covers approximately the entire surface area. The downiness is white in color (RHS 155 C).
   Sepal color.—Green (RHS 139 D).
   Fruit skin.—Appearance — Glabrous, and having a blush which is substantially 90% to about 100% uniform in color.
   Fruit skin.—Thickness — Considered thin.
   Skin texture.—Generally — Considering melting and tender.
   Fruit skin.—Color — The fruit over color is red (RHS 46 A). The fruit skin under color is green-yellow (RHS 1 D).
   Fruit skin.—Lenticels — Generally — Present, distinct and evenly distributed. The lenticels average about 13 per square cm of surface area.
   Fruit skin lenticels.—Shape — Round and very small, and having a size of about 0.05 to about 0.1 mm in diameter.
   Fruit skin lenticels.—Color — White. This color is not distinctive of the present variety.
   Fruit core.—Position. — Considered distant.
   Fruit core.—Line — Median in orientation.
   Fruit core.—Shape — Flat conical.
   Fruit core.—Diameter — On average about 3.5 cm.
   Fruit core.—Length — About 2.6 cm.
   Fruit cell.—Generally — Considered tufted. This characteristic is light and irregular.
   Fruit cell.—Shape — Elliptical.
   Fruit cell.—Length — About 1.6 cm.
   Fruit cell.—Width — About 1.1 cm. The wall-to-wall depth averages about 0.4 cm.
   Tube.—Shape — Cone-like.
   Stamen position.—Considered median.
   Fruit axis.—Considered axial and open.
   Seeds.—Numbers — Generally 2, sometimes 1.
   Seed.—Shape — Considered more acute than acuminate.
   Seed.—Length — About 7.8 mm.
   Seed.—Width — 4.5 mm.
   Seed.—Color — Gray-purple (RHS 187 A).
   Flesh.—Generally — Considered very firm, crisp, melting, juicy and mildly sub-acid and further having a mildly apple-like flavor.
   Flesh.—Color — White (RHS 155 D).
   Flesh.—Aroma — Apple-like, and moderate in intensity.
   Date of harvest.—In 2012 Aug. 29.
   Keeping quality.—Considered very good. The variety has been kept up to five months in common storage with no deleterious affects noted. Further the flesh of the present variety resists browning after being exposed to the ambient atmosphere.
   Fruit pressure upon harvesting.—About 21.9 lbs. This is in contrast to the fruit pressure of the fruit produced by the ‘Cripps Pink Cultivar’ (U.S. Plant Pat. No. 7,880) and which is typically 20.1 lbs.; and that of the ‘Honeycrisp’ apple tree (U.S. Plant Pat. No. 7,197) and which is typically 16.5 lbs upon harvesting. When this same characteristic is measured, two and one-half months following harvesting, the present variety has a fruit pressure of about 20.8 lbs.
   Brix.—Upon harvesting, the present variety has a brix of typically about 14.6°. This is in contrast to the brix as seen upon harvesting of the ‘Cripps Pink Cultivar’ (U.S. Plant Pat. No. 7,880) and which is typically about 13.8°; and that of the ‘Honeycrisp’ apple tree (U.S. Plant Pat. No. 7,197), and which produces fruit having a brix of about 14.8°. After experiencing common storage for two and one-half months, the brix of the present variety remains 14.6°.
   pH.—Upon harvesting, the fruit of the present variety, has a pH of about 3.46. This is in contrast to the fruit produced by the ‘Cripps Pink Cultivar’ (U.S. Plant Pat. No. 7,880), and which has a typical pH, upon harvesting, of 3.32. Still further, the pH of the fruit of the ‘Honeycrisp’ apple tree (U.S. Plant Pat. No. 7,197), upon harvesting, is typically about 3.38. Following storage for about 10 weeks, the fruit of the present variety typically displays a pH of about 3.50.
   Acid content.—Upon harvesting, the present variety typically has an acid content of about 7.4 grams per liter. This is in contrast to the acid content of the fruit produced by the ‘Cripps Pink Cultivar’ (U.S. Plant Pat. No. 7,880), and which typically displays an acid content of 8.4 grams per liter. Still further, the fruit produced by the ‘Honeycrisp’ apple tree (U.S. Plant Pat. No. 7,197) upon harvest, has an acid content of 4.50 grams per liter. Upon storage for a period of two and one-half months, the present variety displays an acid content of about 6.30 grams per liter.
   Pollination.—Generally speaking, any diploid apple tree of the same bloom period is acceptable.
   Fruit use.—Fresh dessert. The present fruit has excellent eating qualities and is considered to be snappy, juicy, sweet and melting.
   Disease and insect resistance.—The present variety is considered to be susceptible to all insects and diseases found in the region of Central Washington State. The present fruit does not exhibit any physiological disorders on the tree or during cold storage for normal storage durations. The present variety has not been subjected either to drought or other adverse environmental conditions to determine its suitability for those conditions.

Although the new variety of apple tree herein denominated as ‘Regal 11-79’ possesses the described characteristics when grown under the ecological conditions prevailing in Grant County, Washington, it is to be understood that variations of the usual magnitude and characteristics incident to changes in growing conditions, fertilization, pruning and pest control are to be expected.

Having thus described and illustrated my new variety of apple tree, what I claim is new and desire to secure by plant Letters Patent is:

1. A new and distinct variety of apple tree, substantially as illustrated and described, and which is characterized principally as to novelty by producing a small, attractively colored, and flavorful apple which is mature for harvesting and shipment approximately the last week in August under the ecological conditions prevailing in Grant County, Washington.
